(19) United States
(12) Patent Application Publication
(10) Pub. No.: US 2023/0035208 A1
(43) Pub. Date: Feb. 2, 2023

(54) CLINICAL TRIAL/PATIENT FOLLOW-UP PLATFORM

(71) Applicant: Hawthorne Effect, Inc., Alamo, CA (US)

(72) Inventor: Jodi J. Akin, Alamo, CA (US)

(73) Assignee: Hawthorne Effect, Inc., Alamo, CA (US)

(21) Appl. No.: 17/958,567

(22) Filed: Oct. 3, 2022

Related U.S. Application Data
(63) Continuation of application No. 15/312,702, filed on Nov. 21, 2016, now abandoned.
(60) Provisional application No. 62/000,802, filed on May 20, 2014.

(51) Int. Cl. G16H 10/20; G16H 10/60; G16H 40/63

(52) U.S. Cl. CPC G16H 10/20; G16H 10/60; G16H 40/63

(57) ABSTRACT

A method and system are provided to improve the implementation of clinical trials by improving patient interaction and follow-up, data collection, and information processing in order to lower costs and cycle time of clinical trials. The invention and methods embodied produce a non-biased, more positive experience (easier, efficient) and reduce fragmentation of the follow-up process for clinical trial follow-up for patients and health care providers or trial team who are tasked with follow-up activities and thereby improve the overall follow-up rate and completeness of data collected. Embodiments of the inventive method leverage the concept called “The Hawthorne Effect”. The Hawthorne Effect is a psychological phenomenon that produces an improvement in human behavior or performance as a result of increased attention or observation.

### RELATED APPLICATIONS

This application is a continuation of U.S. application Ser. No. 15/312,702, filed Nov. 21, 2016, now abandoned, that is a U.S. National Phase of PCT/US2015/031718, filed May 20, 2015, which in turn claims priority benefit of U.S. Provisional Application Ser. No. 62/000,802, filed May 20, 2014, the contents of which are hereby incorporated by reference.

### FIELD OF THE INVENTION

The present invention in general relates to the field of clinical studies and in particular to a platform for implementing clinical trials and tracking patient response to interventions, procedures, drugs and devices.

### BACKGROUND OF THE INVENTION

Clinical studies (trials) are prospective biomedical or behavioral research studies on human subjects that are designed to answer specific questions about biomedical or behavioral interventions, generate safety and efficacy data and/or collect general health information for observing trends in clinical outcomes over time. Examples of clinical trials include trials for novel vaccines, drugs, treatments, devices, behaviors or new ways of using known interventions. Clinical trials are conducted only after satisfactory information has been gathered that satisfies health authority and ethics committee approval in the country where approval of the therapy is sought.

Depending on product, procedure or medical intervention type and development stage, investigators initially enroll volunteers and/or patients into small pilot studies, and subsequently conduct progressively larger scale comparative studies. As positive safety and efficacy data are gathered, the number of patients typically increases. Clinical trials can vary in size, and can involve single or multiple research entities in one or multiple countries.

Clinical trials can be quite costly, with the costs of clinical trials having nearly doubled in the last decade. In fact, a full series of trials may cost hundreds of millions of dollars with the burden of paying usually borne by the sponsor, which may be a governmental organization, an investor or investment fund, a pharmaceutical, biotechnology or medical device company. In addition, hospitals may pay to participate in a clinical trial in order to be first in line for treatment options for patients. When the required support exceeds the sponsor's capacity, the trial may be managed by an outsourced partner, such as a contract research organization or an academic clinical trials unit.

Besides the great costs associated with clinical trials, clinical trials may be quite lengthy and may last several years. There are many reasons why clinical trials can be so lengthy including that for chronic conditions such as cancer, it takes months, if not years, to see if a cancer treatment has an effect on a patient, or if there is a potentially late safety signal not detected in the early follow-up period. For drugs, devices, or treatments that are not expected to have a strong effect (meaning a large number of patients must be recruited to observe ‘any’ effect), recruiting enough patients to test the drug's effectiveness (i.e., getting statistical power) can take several years. Furthermore, only certain people who have the target disease condition are eligible to take part in each clinical trial. Researchers who treat these particular patients must participate in the trial. Then researchers must identify the desirable patients and obtain consent from them or their families to take part in the trial. In fact, trial design features such as patient selection criteria or desired outcome variables are sometimes limited in anticipation of potential poor follow-up or inability to obtain necessary or interpretable/high quality information in the follow-up period. Finally, the biggest barrier to completing studies is the shortage of people who take part. All drug and many device trials target a subset of the population, meaning not everyone can participate. Some drug trials require patients to have unusual combinations of disease characteristics. It is a challenge to find the appropriate patients and obtain their consent, especially when they may receive no direct benefit (because they are not paid, the study drug is not yet proven to work, or the patient may receive a placebo). In the case of cancer patients, fewer than 5% of adults with cancer will participate in drug trials.

Additional problems associated with carrying out clinical trials on a timely and effective basis include lost documentation or incomplete or poor patient follow-up, incomplete tests, poor quality of collected data, lack of sufficient evidence to drive decisions, which all lead to frustration and impedes timely approvals of novel therapies, which slows innovation and adds costs to sponsors, regulators, and ultimately consumers and the healthcare system overall. In addition, delayed and ineffective trials lower investment returns to sponsors and investors.

As a result of the protracted enrollment-follow-up-data analysis and reporting of clinical study results as described above, it can and has been the case that by the time the results of such studies are known, newer iterations of the study drug, device or procedure has been introduced-rendering the study arm obsolete or less relevant and the cycle begins again. Thus, while clinical trials can be an effective tool there exists a need for a method and system to improve the implementation of clinical trials by improving the quality and quantity of patient interaction and follow-up, data collection, and information processing in order to lower costs and cycle time of clinical trials and improve impact, timing and proliferation of new therapies for patients.

### SUMMARY OF THE INVENTION

An automated method for implementing a clinical trial is provided, the method including: providing a graphical user interface (GUI) from a clinical trial platform; receiving a request for registration of a clinical trial from a sponsor; generating and assigning a unique identifier for the clinical trial; receiving inputs from the sponsor for follow-up visit timelines and relevant protocol requirements that are programmed into a scheduler provided by the platform; configuring a doctor bag based on the received inputs from the sponsor; providing a customized program that integrates with a clinical database of the sponsor; providing a widget as a patient is enrolled via the platform in the clinical trial; matching each of the enrolled patients with a clinical trial worker based on an algorithm in a main database of the platform; and receiving patient data for the patients generated during clinical trial worker visits with the patient.

A system for clinical trials, the system including: a server connected via a network to user devices for use by a series of users; a memory system in electrical communication with the server containing a machine readable medium having stored thereon one or more sequences of instructions which, when executed by a processor, cause a method to be carried out, the method including: providing a graphical user interface (GUI) from a clinical trial platform; receiving a request for registration of a clinical trial from a sponsor; generating and assigning a unique identifier for the clinical trial; receiving inputs from the sponsor for follow-up visit timelines and relevant protocol requirements that are programmed into a scheduler provided by the platform; configuring a doctor bag based on the received inputs from the sponsor; providing a customized program that integrates with a clinical database of said sponsor; providing a widget as a patient is enrolled via said platform in the clinical trial; matching each of the enrolled patients with a clinical trial worker based on an algorithm in a main database of said platform; and receiving patient data for the patients generated during clinical trial worker visits with the patient.

### BRIEF DESCRIPTION OF THE DRAWINGS

The present invention is further detailed with respect to the following drawings. These figures are not intended to limit the scope of the present invention but rather illustrate certain attributes thereof.

FIG. 1 is a block diagram showing the value created to stakeholder by embodiments of the invention;

FIG. 2 is a flowchart of a method and workflow of a trial using the clinical trial platform according to an embodiment of the invention; and

FIG. 3 is a schematic diagram illustrating an overall view of communication devices, computing devices, and mediums for implementing embodiments of the invention.

### DESCRIPTION OF THE INVENTION

The present invention has utility as a method and system to improve the implementation of clinical trials by improving patient interaction and follow-up, data collection, and information processing in order to lower costs and cycle time of clinical trials. The invention and methods embodied may produce a non-biased, more positive experience (easier, efficient) and reduce fragmentation of the follow-up process for clinical trial follow-up for patients and health care providers or trial team who are tasked with follow-up activities and thereby improve the overall follow-up rate and completeness of data collected. Embodiments of the inventive method leverage the concept called “The Hawthorne Effect”. The Hawthorne Effect is a psychological phenomenon that produces an improvement in human behavior or performance as a result of increased attention or observation. Anticipated behavioral improvements include but are not limited to the completeness and quality of follow-up, motivation for patients to participate in follow-up examinations, quality of the healthcare provider interactions with patients, timeliness and interpretability of data analyses and improvement of regulatory review cycles etc. It is appreciated that the present invention is also of value in various other contexts that include: Patient follow-up from hospital discharge such as a home visit or multiple to ensure patient understands discharge instructions, educational follow-up and collection of any follow-up info necessary, video documentation or connection with primary team; Post newborn baby visits; Student physicals; Executive physicals; Athletic physicals; and Patients with communicable diseases or immunosuppression where hospitals are dangerous places; and dietary program follow-up.

The tracking of patient response to medical or surgical treatments is essential to the entire healthcare ecosystem, and embodiments of the invention streamline and transform patient follow-up and follow-through by integrating technological innovations with the tradition of personalized medicine. Embodiments of the inventive process provide a centralized platform to which a sponsor of a clinical trial registers the trial, and allows the sponsor to create a customized “Doctor's Bag” that is fitted with study appropriate diagnostics, data entry devices and linkages to the study database. Diagnostics and data entry devices operative herein illustratively include vital sign and biometric response sensors, such as sphygmomanometry, electrocardiogram, electroencephalogram, a respirometer, otoscope, opthalmoscope, imaging devices such as echocardiograms, vascular dopplers, scanners, serum blood tests, or a combination thereof; a chemical sensors such as a glucometer, pulse oximetry, or a combination thereof. Additionally survey instruments such as quality of life surveys, patient experience surveys, activities of daily living, neurological, behavioral, psychosocial instruments are routinely including in clinical studies. These diagnostics and surveys do not have a common platform for integrating the patient results into the clinical study databases and there are a variety of methods used to collect data including paper and pen/pencil, computer output from lab equipment, etc. The linkages to the study database between the aforementioned diagnostics and data entry devices include Wi-Fi protocols, radiofrequency identification tags, physically wiring, Internet communication, video (moving) and still image documentation and combinations thereof.

In embodiments of the inventive process, Patients or study participants may elect for additional evaluations. A visit scheduler links appropriate medical professional to patients or study participants by geography and other visit requirements, such as language capabilities and allows patient visits to be conducted at home and remote from the sponsor site. Participating patient visit results are immediately available electronically to all identified stakeholders (i.e., sponsor, regulator, patient, investor, etc.) in the clinical trial or study via the linkage provided to the patient or similar interface to equipment associated with the visiting clinician.

In embodiments of the inventive method and system clinicians carrying out patient visits and data gathering may be independent contractors whose credentials, location coordinates, language skills and availability are vetted through various applications and through the central operations of the system. Applications (app) available in the system include a patient app, a sponsor app, and a clinical team app. Clinicians within the inventive system are each equipped with a doctor bag for carrying out the patient visits and data collection. The doctor bag includes portable diagnostics and a portable communications and computing device, such as but not limited to a tablet device. The portable communications and computing device may be configured with audio and video capabilities for communications with other clinicians and primary team members. Clinicians may also use their portable devices for data entry level access to medical databases related to the trial they are gathering data for, as well as a reporting tool in a virtual environment.

Within the inventive system, field clinicians may rely on a primary team of licensed, credentialed, and trained specialists including physicians by specialty, physician assistants, nurses, physiotherapists, image specialists, and other specialties within an on-line social network environment. The social network for medical professionals provides opportunities for professional development, skill expansion, continuing education, and professional independence.

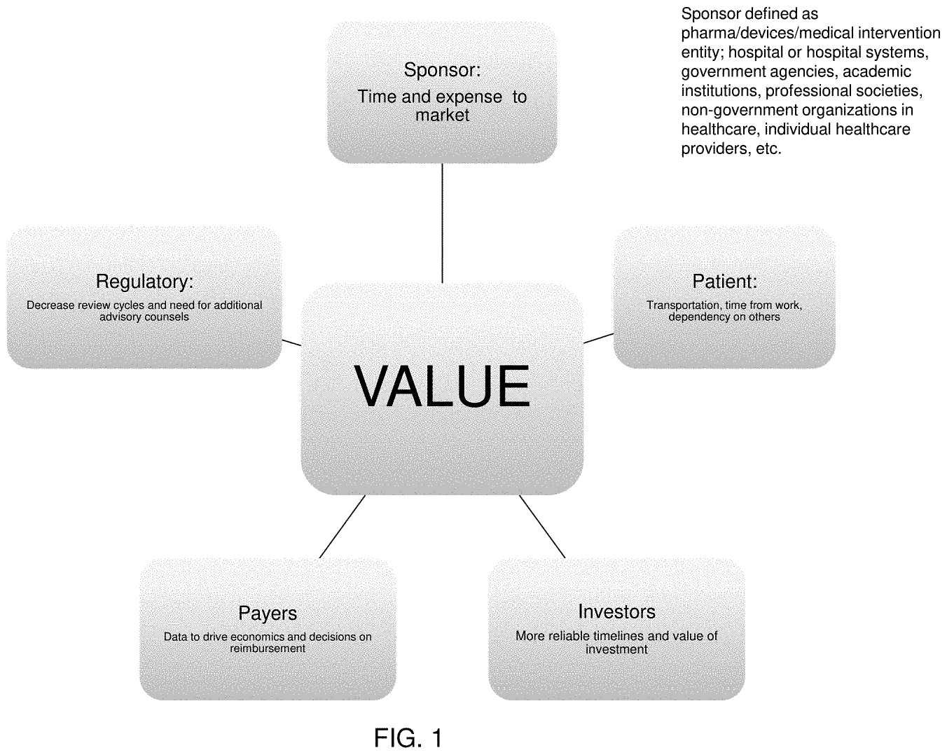

FIG. 1 is a graphical representation of the value creation gained through embodiments of the system and method for carrying out clinical trials. Embodiments of the invention create value by making visits to trial participants timely, complete and cost efficient. Improved and more accurate data gathering decreases delays in regulatory review, thereby speeding time to market of the treatment or drug to market and furthers the pace of innovation and quality of life. Benefits provided to stakeholders include: Sponsor—saved time and expense to market; Patient—saved transportation costs, time from work, dependency on others for help to attend clinical trials; Regulatory—decreased review cycles and need for additional advisory councils; Payers—provides data to drive economics and decisions on reimbursement; Investors—more reliable, timeliness of information to make informed and value of investments.

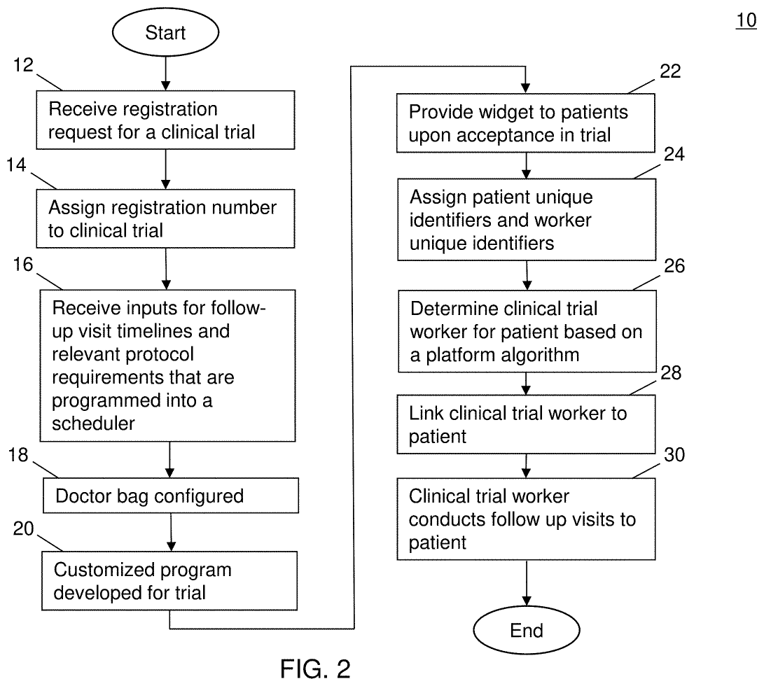

A process and workflow chart 10 for using the embodiments of the inventive clinical trial platform is illustrated in the FIG. 2 as follows. A sponsor (pharma, device manufacturer, lab group, hospital group/system, healthcare delivery system, government agency, or the like) registers a clinical study with details and parameters via a graphical user interface (GUI) with the clinical trial platform (Block 12). The platform in response to the registration of the clinical trial assigns a unique identifier to the trial (Block 14), and under the unique identifier accepts inputs from the sponsor for follow-up visit timelines and relevant protocol requirements that are programmed into a scheduler provided by the platform (Block 16). Relevant diagnostic tools are integrated into the doctor bag based on the sponsor inputs and study parameters, and custom programmed for patient study identifier, HIPPA compliant de-identification and adapted to the study data base case report form (Block 18). The platform develops and provides a customized program designed for use on a mobile computing and communication device, such as a tablet, that integrates with a sponsor's clinical database (Block 20). As patients are enrolled and accepted into the sponsor's clinical trial database by the participating site or sponsor, the platform provides a patient data collection device (USB, RF tag, swipe card, mini SD card, flash memory) that can be plugged into a mobile phone, tablet, laptop or desktop computer of the patient (Block 22). The patient data collection device incorporates a chip with information about the clinical trial and is designed to capture or have patient specific information inputted and capable to be transferred to another data collection repository via cloud, electronic transfer or other means of input (basic demographics, medical history, baseline study data, specific needs or preferences, language needs, etc.). Patient generated data can also be inputted (by patient with capability of audit trails and verification systems. The patient data collection device is programmed by the platform in cooperation with the sponsor. It is appreciated that such programming is completed either prior to installation with the patient or remotely programmed via the Internet or an intranet to communication with the sponsor. In response to the patient enrollment, an algorithm in the main database of the platform matches clinical trial workers (clinician contractors) availability, credentials and geographical location with the patient visit requirements, language needs and other possible preferences (Block 24). The platform then links an appropriate available contractor in the area to the patient, and the contractor or systems makes the introductory handshake via email, instant message (IM), phone call, etc., and the matched parties take on the scheduling and interactions to prepare for the visits (Block 26). Once a visit has been scheduled, the contractor, using their own means of transportation, but equipped with the platform provided doctor's bag, professional wear and essential visit conduct training conducts the follow-up visits for the clinical trial the patient is registered for (Block 28). Included in the patient visit is the inputting or data transfer activities to ensure the patient data is applied to the sponsor databases as quickly as possible. During the visit, the contractor can arrange a video link to the patient's primary care team (if required, or requested or approved by the patient), or may tape the interview for the primary team files. The primary team will also receive the data from the visit either through cloud based technology, chip storage, or direct linkages to the hospital or platform system databases. Data entry and transfers of any sort can be audited.

The clinical trial platform may allow patients and or family members to rate the performance and quality of the contractor visit for ongoing performance metrics of the contractors. The platform may provide contractors with continuous educational forums and opportunities to expand skill sets. The platform can provide a patient with access to their contractor and the trial sponsor for questions/concerns, or new information. The platform may provide patients with an ongoing, updated graphically depicted progress chart of their progress through the study and in relationship to their overall medical history. Patients may also be able to elect for additional tests or surveys, or educational consultations on health, wellness, etc., when the visits are scheduled. (a menu of options will appear during the interactive scheduling process on the schedule menu of the graphical user interface (GUI) of the platform.

The inventive widget provided to patients by the clinical trial platform has applications stored that allow a medical history on a chip to be viewed on a screen (with the medical team, or projected onto a wall skin, or touch screen for patients to interact—adding key medical history milestones, like, birth, children, hospitalizations). Patients may be able to see test results, or progress for example with nutrition, weight management, cholesterol, glucose management, etc.

The widget may include an initial patient survey to acquire basic demographic and medical milestone info, but with more bio info such as where born, what languages spoken, cultural religious preferences, attitudes about healthcare systems/providers, etc. for best match of contractor personnel for interaction with the patient. The patient may be able to keep a diary about their health on the platform via the widget. In an embodiment the use of and measurements obtained from personalized wearable health devices and monitors can be integrated with the applications stored on the widget.

In specific embodiments of the clinical trial platform, a cumulative document that is useful for each clinical trial visit, but also when traveling or in a new medical clinical environment to familiarize new medical team member with patient history and subtle important psycho social factors (language, culture, personality type, etc.)

In specific embodiments, the doctor bag used by the contractor clinicians may be equipped with devices and methods to measure patient experience during the home visit (biometrics such as brain waves, heart rate, comfort level, etc.)

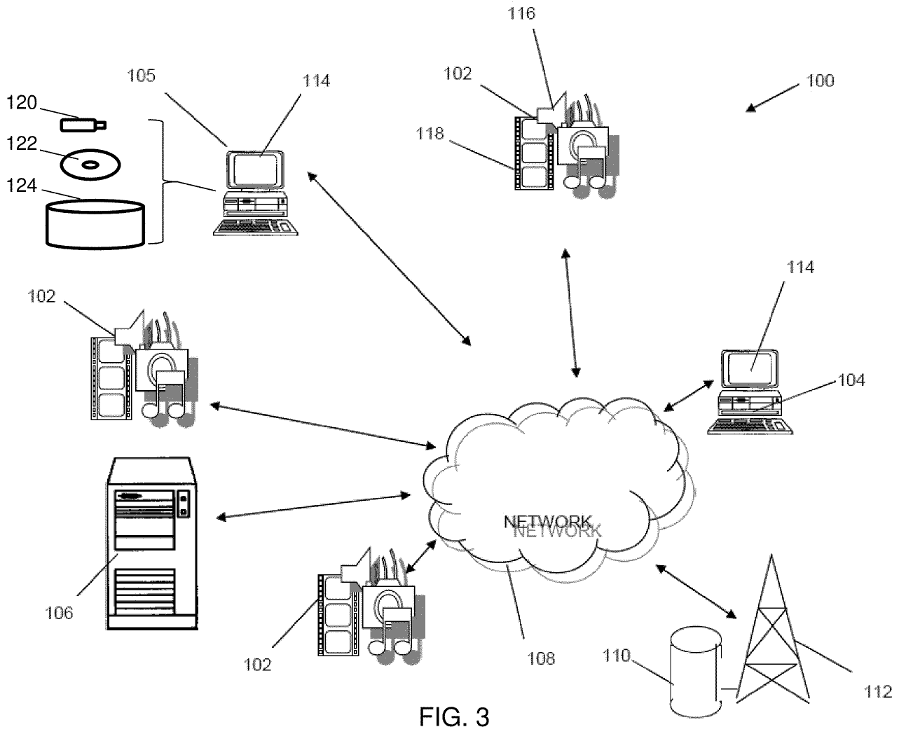

FIG. 3 is a schematic diagram illustrating an overall view of communication devices, computing devices, and mediums for implementing the clinical trial platform according to embodiments of the invention.

The system 100 includes multimedia devices 102 and desktop computer devices 104 configured with display capabilities 114 and processors for executing instructions and commands. The multimedia devices 102 are optionally mobile communication and entertainment devices, such as cellular phones, tablets, and mobile computing devices that in certain embodiments are wirelessly connected to a network 108. The multimedia devices 102 typically have video displays 118 and audio outputs 116. The multimedia devices 102 and desktop computer devices 104 are optionally configured with internal storage, software, and a graphical user interface (GUI) for carrying out elements of the platform according to embodiments of the invention. The network 108 is optionally any type of known network including a fixed wire line network, cable and fiber optics, over the air broadcasts, satellite 120, local area network (LAN), wide area network (WAN), global network (e.g., Internet), intranet, etc. with data/Internet capabilities as represented by server 106. Communication aspects of the network are represented by cellular base station 110 and antenna 112. In a preferred embodiment, the network 108 is a LAN and each remote device 102 and desktop device 104 executes a user interface application (e.g., Web browser) to contact the server system 106 through the network 108. Alternatively, the remote devices 102 and 104 may be implemented using a device programmed primarily for accessing network 108 such as a remote client. The network 108 may be accessed by the user during a recovery operation to reach online help.

The software for the platform, of embodiments of the invention, may be resident on a widget (USB thumb, flash or other storage drive) 120, CD or DVD 122, or an external hard drive 124 for connection to desktop or laptop computers 104, or stored within the server 106 or cellular base station 110 for download to an end user. Server 106 may implement a cloud-based service for implementing embodiments of the platform with a multi-tenant database for storage of separate client data for each independent trial being carried out on the platform.

The foregoing description is illustrative of particular embodiments of the invention, but is not meant to be a limitation upon the practice thereof. The following claims, including all equivalents thereof, are intended to define the scope of the invention.

1. An automated method for implementing a geographically dispersed clinical trial, said method comprising:
providing a clinical trial platform having a graphical user interface (GUI) that is accessible from a plurality of geographically dispersed computing devices;
receiving in said clinical trial platform via the GUI on a first computing device of the plurality of computing devices a request for registration of a clinical trial from a sponsor having a primary geographic location;
generating and assigning by said clinical trial platform a unique identifier for said clinical trial;
receiving in said clinical trial platform inputs from the sponsor via the GUI for follow-up visit timelines and relevant protocol requirements;
automatically programming by said clinical trial platform the follow-up visit timelines and relevant protocol requirements into a scheduler provided by said clinical trial platform;
configuring a plurality of doctor bags based on the received inputs from the sponsor, each of said plurality of doctor bags comprising clinical trial appropriate portable diagnostics, healthcare survey instruments, one of said geographically dispersed computing devices;
providing by said clinical trial platform a customized program that integrates with the clinical database of said clinical trial platform, said customized program being accessible via the GUI from the plurality of geographically dispersed computing devices;
receiving in said clinical trial platform via the GUI information regarding a plurality of enrolled clinical trial patients and storing said information regarding the plurality of enrolled clinical trial patients in said clinical trial database;
receiving in said clinical trial platform via the GUI information regarding a plurality of approved clinical trial workers and storing said information regarding the plurality of approved clinical trial workers in said clinical trial database;
matching by the clinical trial platform each of said plurality of enrolled patients with one of said plurality of approved clinical trial workers based on an algorithm in a main database of said clinical trial platform;
supplying one of said plurality of doctor bags to each to said plurality of approved clinical trial workers that is matched with one of said plurality of enrolled clinical trial patients;
automatically scheduling by a visit scheduler of the clinical trial platform a plurality of visits for each of said plurality of enrolled patients at a unique location for each of said plurality of enrolled patients that is geographically remote from the primary geographic location of said sponsor;
receiving by the clinical trial platform via the GUI of one of the plurality of geographically dispersed computing devices patient data for each of said plurality of enrolled patients at each of the scheduled visits, wherein the patient data is collected by one of said plurality of approved clinical trial workers using the clinical trial appropriate portable diagnostics and healthcare survey instruments of the doctor bag at the unique location for each of said plurality of enrolled patients that is geographically remote from the primary geographic location of said sponsor
providing immediate, real-time access to the received patient data in a HIPPA compliant de-identified form via the clinical trial platform that is accessible from said plurality of geographically dispersed computing devices.

2. The method of claim 1 wherein said sponsor is a pharmaceutical company, a medical device manufacturer, a lab group, a hospital, a healthcare delivery system, an academic group, an individual healthcare provider, a government or non-government agency or a combination thereof.

3. The method of claim 1 further comprising providing a widget to each of said plurality of enrolled patients, where said widget comprises a chip with a set of information about said clinical trial, said chip programmed by said clinical trial platform in cooperation with said sponsor, and where said chip is configured to capture or have patient specific information inputted via a set of applications, said applications allowing for patient interactions and progress monitoring, as well as integration with personalized wearable health devices and monitors.

4. The method of claim 4 wherein said widget is configured to be plugged into a mobile phone, tablet, laptop or desktop computer of the patient.

5. The method of claim 1 wherein said information regarding the plurality of enrolled clinical trial patients comprises basic demographics, medical history, baseline study data, specific needs or preferences, and language needs.

6. The method of claim 1 wherein said algorithm for matching said plurality of enrolled patients with one of said plurality of approved clinical trial workers bases the matching on availability, credentials and geographical location with the patient visit requirements, language needs and other possible preferences.

7. The method of claim 1 wherein said clinical trial platform is configured for said patient to rate the performance and quality of said clinical trial worker.

8. The method of claim 1 wherein said clinical trial platform provides said plurality of approved clinical trial workers with continuous educational forums and opportunities to their expand skill sets.

9. The method of claim 1 wherein said clinical trial platform provides training/education of said plurality of approved clinical trial workers, said plurality of enrolled patients and families of said plurality of enrolled patients; and
wherein said training/education comprises healthcare state, treatment, manufacturers products, procedures, instructions for use, and study protocol information.

10. The method of claim 1 wherein said clinical trial platform provides each of said plurality of enrolled patients with an ongoing, real-time graphically depicted progress chart of their progress through the trial and in relationship to their overall medical history.

11. A system for clinical trials, said system comprising:
a server connected via a network to user devices for use by a series of users;
a memory system in electrical communication with said server containing a machine readable medium having stored thereon one or more sequences of instructions which, when executed by a processor, cause a method to be carried out, the method comprising:
providing a clinical trial platform having a graphical user interface (GUI) that is accessible from a plurality of geographically dispersed computing devices;
receiving in said clinical trial platform via the GUI on a first computing device of the plurality of computing devices a request for registration of a clinical trial from a sponsor having a primary geographic location;
generating and assigning by said clinical trial platform a unique identifier for said clinical trial;
receiving in said clinical trial platform inputs from the sponsor via the GUI for follow-up visit timelines and relevant protocol requirements;
automatically programming by said clinical trial platform the follow-up visit timelines and relevant protocol requirements into a scheduler provided by said clinical trial platform;
configuring a plurality of doctor bags based on the received inputs from the sponsor, each of said plurality of doctor bags comprising clinical trial appropriate portable diagnostics, healthcare survey instruments, one of said geographically dispersed computing devices;
providing by said clinical trial platform a customized program that integrates with the clinical database of said clinical trial platform, said customized program being accessible via the GUI from the plurality of geographically dispersed computing devices;
receiving in said clinical trial platform via the GUI information regarding a plurality of enrolled clinical trial patients and storing said information regarding the plurality of enrolled clinical trial patients in said clinical trial database;
receiving in said clinical trial platform via the GUI information regarding a plurality of approved clinical trial workers and storing said information regarding the plurality of approved clinical trial workers in said clinical trial database;
matching by the clinical trial platform each of said plurality of enrolled patients with one of said plurality of approved clinical trial workers based on an algorithm in a main database of said clinical trial platform;
supplying one of said plurality of doctor bags to each to said plurality of approved clinical trial workers that is matched with one of said plurality of enrolled clinical trial patients;
automatically scheduling by a visit scheduler of the clinical trial platform a plurality of visits for each of said plurality of enrolled patients at a unique location for each of said plurality of enrolled patients that is geographically remote from the primary geographic location of said sponsor;
receiving by the clinical trial platform via the GUI of one of the plurality of geographically dispersed computing devices patient data for each of said plurality of enrolled patients at each of the scheduled visits, wherein the patient data is collected by one of said plurality of approved clinical trial workers using the clinical trial appropriate portable diagnostics and healthcare survey instruments of the doctor bag at the unique location for each of said plurality of enrolled patients that is geographically remote from the primary geographic location of said sponsor
providing immediate, real-time access to the received patient data in a HIPPA compliant de-identified form via the clinical trial platform that is accessible from said plurality of geographically dispersed computing devices.

12. The system of claim 11 wherein said sponsor is a pharmaceutical company, a medical device manufacturer, a lab group, a hospital, a healthcare delivery system, an academic group, an individual healthcare provider, a government or non-government agency or a combination thereof.

13. The system of claim 11 wherein said doctor bag further comprises: trial appropriate portable diagnostics, portable data entry devices and linkages to the study database.

14. The system of claim 1 further comprising a widget provided to each of said plurality of enrolled patients, where said widget comprises a chip with a set of information about said clinical trial, said chip programmed by said clinical trial platform in cooperation with said sponsor, and where said chip is configured to capture or have patient specific information inputted via a set of applications, said applications allowing for patient interactions and progress monitoring, as well as integration with personalized wearable health devices and monitors.

15. The system of claim 14 wherein said widget is configured to be plugged into a mobile phone, tablet, laptop or desktop computer of the patient.

16. The system of claim 11 wherein information regarding the plurality of enrolled clinical trial patients comprises basic demographics, medical history, baseline study data, specific needs or preferences, and language needs.

17. The system of claim 11 wherein said algorithm for matching said plurality of enrolled patients with one of said plurality of approved clinical trial workers bases the matching on availability, credentials and geographical location with the patient visit requirements, language needs and other possible preferences.

18. The system of claim 11 wherein said clinical trial platform is configured for said patient to rate the performance and quality of said clinical trial worker.

19. The system of claim 11 wherein said clinical trial platform provides said plurality of approved clinical trial workers with continuous educational forums and opportunities to their expand skill sets.

20. The system of claim 11 wherein said clinical trial platform provides each of said plurality of enrolled patients with an ongoing, real-time graphically depicted progress chart of their progress through the trial and in relationship to their overall medical history.
